(19) United States
(12) Patent Application Publication
(10) Pub. No.: US 2012/0138495 A1
(43) Pub. Date: Jun. 7, 2012

(54) UNIVERSAL SURGICAL FASTENER STERILIZATION CADDY

(76) Inventors: Cary A. Bettenhausen, Indianapolis, IN (US); Todd E. Bettenhausen, Indianapolis, IN (US); Thomas A. Deal, Indianapolis, IN (US)

(21) Appl. No.: 13/372,569

(22) Filed: Feb. 14, 2012

Related U.S. Application Data
(63) Continuation-in-part of application No. 12/615,020, filed on Nov. 9, 2009, now Pat. No. 8,162,138.

(51) Int. Cl. A61B 19/02; B65D 25/02; B65D 85/24

(52) U.S. Cl. 206/339; 206/438

(57) ABSTRACT

A universal surgical screw caddy. An enclosure includes a cover secured to a frame. A flexible sheet is located between and affixed to the cover and frame. A plurality of holes formed in the cover allow the surgical screws to be inserted into the caddy past a plurality of movable portions of the flexible sheet releasably holding the screws in the caddy.

This application is a continuation-in-part of U.S. patent application Ser. No. 12/615,020, filed Nov. 9, 2009 which is hereby incorporated by reference.

### BACKGROUND OF THE INVENTION

1. Field of the Invention

The present invention relates generally to the field of surgical screw racks.

2. Description of the Prior Art

Surgical fasteners, such as surgical screws, must be sterilized, stored and be convenient for use during surgery. A variety of surgical screw racks have been devised having a plurality of holes into which a separate surgical screw is removably positioned. The racks may include covers movably mounted thereto to prevent a sterilized screw from accidentally falling from the rack. The covers may be slidably or hingedly mounted to the rack. In the event the rack is dropped, the screws might become dislodged thereby requiring complete re-sterilization.

Disclosed herein is a caddy for removably holding a plurality of surgical fasteners. The caddy does not include a movable cover to hold the fasteners and thus the screws are more accessible and convenient for use during surgery. Further, the shanks of the fasteners are positioned within the caddy whereas the fastener heads protrude above the caddy allowing for easy grasping to pull the particular fastener from the caddy. The caddy with mounted screws may be inserted directly into sterilization without requiring a separate container. Conventional fabric may be wrapped around the caddy for the sterilization.

Internal passages within the caddy allow for the sterilization fluid that enters the caddy to readily exit the caddy providing for a dry and sterile fastener. In the event the caddy disclosed herein is accidentally dropped, the fasteners are still held within the caddy by a plurality of releasable flaps.

### SUMMARY OF THE INVENTION

One embodiment of the present invention is the combination of a surgical implant holder for holding surgical implants having shanks and side shank surfaces. A flexible sheet includes a plurality of flaps with proximal ends integral with the sheet and free distal ends. The flaps are arranged in groups defining areas in the sheet through which shanks of surgical implants may be extended with the flaps having side contact surfaces engageable with the shanks to releasably hold the shanks. A cover is mounted to the frame and positioned outwardly of the flexible sheet limiting outward movement of the flexible sheet when the shanks are withdrawn from the holders.

### BRIEF DESCRIPTION OF THE DRAWINGS

FIG. 1 is a perspective view of an embodiment of a surgical caddy having a plurality of surgical screws removably mounted thereto.

FIG. 2 is a cross-sectional view taken along a line of FIG. 1 and viewed in the direction of the 2-2 arrows with the surgical screws removed from the caddy.

FIG. 3 is a cross-sectional view taken along a line of FIG. 2 and viewed in the direction of the 3-3 arrows showing one of the surgical screws located within one of the caddy passages.

FIG. 4 is the same view as FIG. 3 only showing an alternate embodiment of the caddy.

FIG. 5 is a perspective view of the preferred embodiment of a surgical caddy.

FIG. 6 is a reduced top view of the caddy of FIG. 5.

FIG. 7 is a side view of FIG. 6.

FIG. 8 is cross-sectional view taken along the line and viewed in the direction of arrows 8-8 of FIG. 7.

FIG. 9 is a top view of the flexible sheet mounted in the caddy of FIG. 5.

FIG. 10 is a side view of FIG. 9.

FIG. 11 is an enlarged fragmentary of a group of flaps shown in the circle 11 of FIG. 9 with the flaps being in a relaxed state.

FIG. 12 is the same view as FIG. 11 only showing a surgical implant inserted between the distal end portions of the flaps which are in a stressed engaged position.

FIG. 13 is an enlarged cross-sectional view taken along the line and viewed in the direction of arrows 13-13 of FIG. 12.

### DESCRIPTION OF THE PREFERRED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

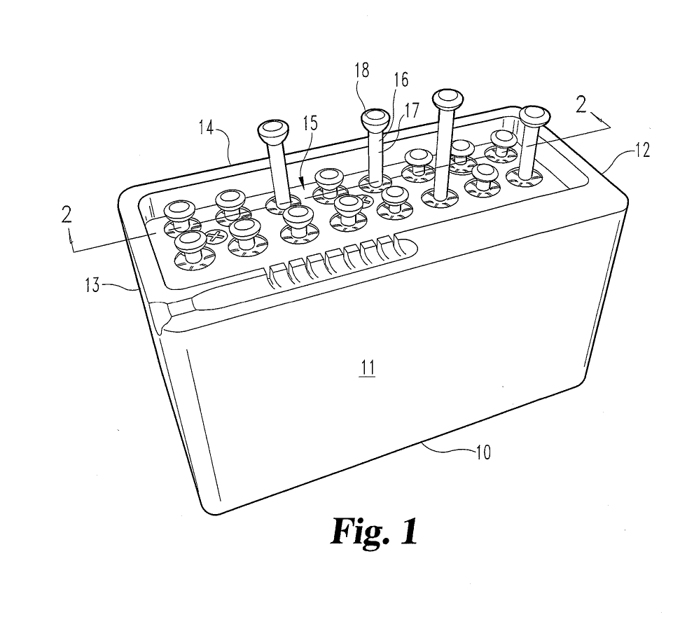

Referring now more particularly to FIGS. 1-3, there is shown an embodiment of a universal surgical fastener caddy to hold surgical fasteners or screws during the sterilization thereof and also when subsequently used during surgery. The caddy 10 includes a box like enclosure having a pair of end walls 12 and 13 joined to side walls 11 and 14. The top end 15 includes a plurality of holes through which the surgical fasteners or screws 16 may be inserted. Surgical screws 16 are commercially available and include a shank 17 having an outside diameter integrally joined to a head 18 also having an outside diameter. A variety of surgical screws are commercially available having a variety of configurations. For example, the shank may have external threads extending along a portion or the entire length of the shank. Likewise, the shanks and heads may be hollow.

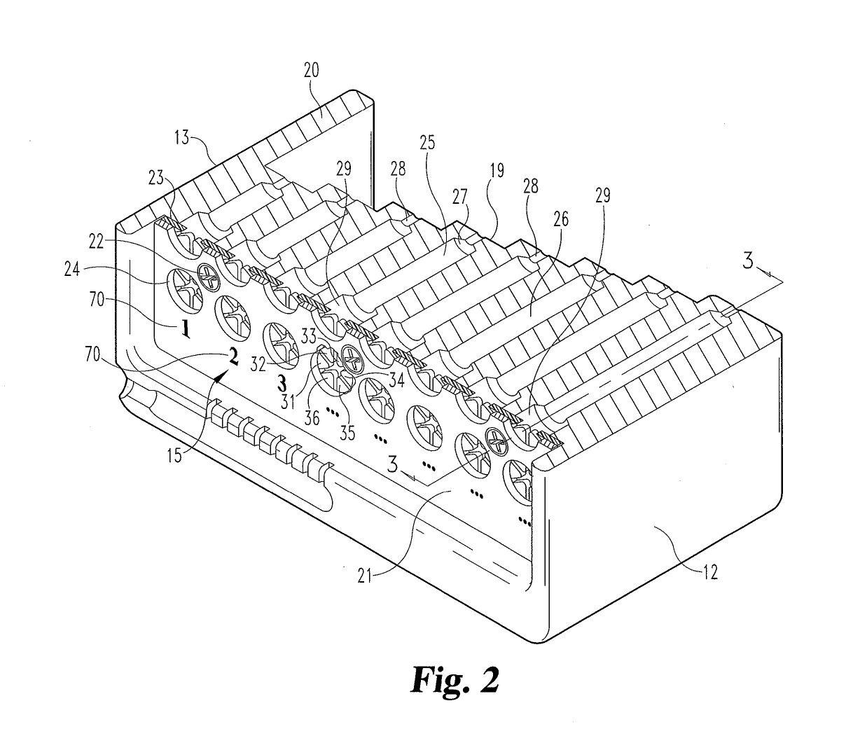

A cross-sectional view of the caddy is shown in FIG. 2 without the surgical screws being inserted therein to more clearly illustrate the construction. In the embodiment of FIG. 2, the frame 20 is in a block configuration and consists of the end walls 12 and 13 integrally joined to the side walls 11 and 14. The bottom 19 of frame 20 may extend between end walls 12 and 13 in a single plane or may be graduated, such as shown in FIG. 2, and extend in a variety of parallel planes to accommodate different lengths of surgical screws.

Frame 20 has an open top 15 that is closed by a rigid cover 21 fixedly secured to frame 20 by a plurality of conventional fasteners 22 extending into the frame. A flexible sheet 23 is parallel to cover 21 and is positioned between frame 20 and cover 21 being held to the frame by fasteners 22. Fasteners 22 have shanks extending through cover 21 and flexible sheet 23 and then threadedly received by frame 20. The head of fastener 22 abuts against the cover 21 thereby securing the cover, sheet and frame together.

Cover 21 is rigid and includes a plurality of screw holes 24 arranged in rows and aligned with passages 25 provided in the main body of frame 20 which are also arranged in rows. Passage 25 will now be described it being understood that an identical description applies to the remaining passages. Passage 25 has a circular cross-section that tapers down at bottom end 27 emptying into a drain hole 28 leading to external of the frame. The top end of passage 25 includes a counter bore portion 29 immediately beneath a hole 24. Typically, the inside diameter of the counter bore portion 29 is the same as the inside diameter of hole 24; however, the present invention includes having a inside diameter of counter bore portion 29 different from the inside diameter of hole 24.

Flexible sheet 23 includes a plurality of flaps or movable portions positioned between each hole 24 and its associated passage 25. In the embodiment shown in FIG. 2, flexible sheet 23 includes six such movable flaps positioned adjacent each hole 24. It is to be understood that the present invention includes more than or less than six movable flaps positioned adjacent each hole 24. As an example, movable flaps 31, 32, 33, 34, 35 and 36 are shown for each hole 24. Each movable flap has a proximal end integrally joined to sheet 23 and a free movable distal end located adjacent the longitudinal axis that extends through hole 24 and passage 25. As a surgical screw is inserted through a hole, the shank of the screw contacts the movable flap thereby forcing the movable flap into a counter bore portion 29 with the shank then extending further into the reduced cylindrical portion 26 of the passage 25 until the tip of the screw contacts the tapered end 27 of the passage that provides a stop surface limiting further extension of the screw into the passage. The depth of the counter bore potion 29 must be deep enough to prevent interference with the tips of the flaps.

As shown in FIG. 2, the passages 25 may have different lengths depending upon the length of the particular surgical screw to be inserted therein. The length of passage 25 is less than the length of the screw shank to be inserted therein so that the bottom end of the screw may contact tapered end 27 while always positioning the head of the screw above cover 21 enabling the user to quickly grasp the head 18 of the surgical screw during surgery.

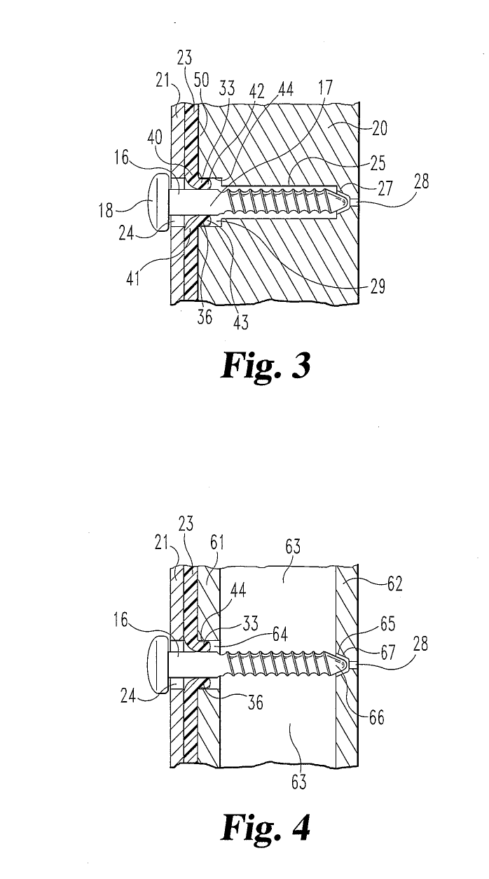

FIG. 3 illustrates a surgical screw 16 inserted into the caddy. The shank 17 of the screw has been inserted through hole 24 contacting the movable flaps of flexible sheet 23 forcing the movable flaps into counter bore 29 while the shank of the screw extends downwardly with the bottom end of the screw contacting the stop surfaces formed by the tapered passage end 27. Instead of forming a tapered passage end 27 to form the stop surfaces contacted by the tip of shank 17, the present invention contemplates and includes using a flat wall, a counter drilled wall, a counter bored wall, or an angled wall to form the stop surfaces leading to or not leading to the drain hole 28 or even not using a wall at all with stop surfaces. The size of counter bore 29 is selected so that the movable flaps are forced against the internal surface of the counter bore 29 by the screw, thereby allowing deflection of the flaps within the counter bore. The distal ends of the flaps are spaced apart a distance less than the diameter of the fastener shank inserted therebetween. Thus, the flaps deflect into the caddy as the fastener shank is inserted and deflected out of the caddy as the fastener shank is pulled from the caddy. As the fastener shank is pulled outwardly and the flaps move from between being deflected inwardly to outwardly relative to the caddy, the flaps will move to a plane generally parallel to the main body of the flexible sheet thereby becoming compressed since the distal ends of the flaps are spaced apart less than the diameter of the shank. Thus, if the caddy is dropped or turned upside down, the screw will not fall from the caddy. On the other hand, the head 18 of the screw is positioned outwardly of cover 21 and may be grasped thereby allowing the user to pull the surgical screw from the caddy.

FIG. 3 illustrates a cross-section of two of the movable portions or flaps 33 and 36 having proximal ends 40 and 41 integrally attached to the flexible sheet whereas the flap distal ends 42 and 43 are free to move into and out of the counter bore. A stop surface 44 is formed by the inside surface of the counter bore allowing the flaps to be deflected as the shank is moved through the counter bore.

During the sterilization of the caddy loaded with surgical screws, the sterilization fluid may enter through hole 24 passing between the flaps and down into passage 25, eventually exiting passage 25 via drain hole 28.

The movable flaps immediately adjacent each hole 24 are arranged to define a hole through the flexible sheet 23 through which the shanks of the surgical screws may be extended with the flaps engageable with the shanks to removably hold the shanks but releasable to enable the user to pull the sterilized screw from the caddy. Cover 21 and the top 50 of frame 20 are rigid in order to limit the movement of the flexible sheet as the surgical screws are inserted and pulled from the caddy. Cover 21 prevents flexible sheet 23 from moving apart from the main body as the screws are pulled from the caddy while allowing the movable flaps to move back to their normal position extending across each passage. On the other hand, the rigid top 50 of frame 20 provides a solid surface limiting movement of the flexible sheet except at the point of the movable flaps as the surgical screws are inserted into each passage.

The counter bore shaped portion include the continuous internal surfaces 44 thereby forming the first stop surfaces which continuously surround the screw shank as it is inserted into the passage. The inside diameter of counter bore 29 is chosen so when coupled with the thickness of the flaps extending into the counter bore in relationship to the outside diameter of the screw shank 17 allows the flaps to be deflected. On the other hand, the diameter of head 18 is sufficiently large as compared to the diameter of screw shank 17 so as to not extend into the counter bore portion being limited by the thickness of the flaps and the internal diameter of the counter bore in relationship to the diameter of the head 18 in the case a relatively short surgical screw is inserted into a relative long passage 25. In the event the proper length of screw is inserted into the proper length of passage, then the bottom tip of the screw contacts the stop surface formed by the tapered passage end 27 ensuring that the head of the screw remains external to the caddy.

The inside diameter of the reduced portion 26 of passage 25 is chosen to be slightly larger than the outside diameter of the screw shank to limit sideways movement of the surgical screw as it is inserted and held within the passage. The inside diameter of the passage is enlarged at the counter bore portion 29 to enable the flexible sheet flap to extend into the counter bore portion.

FIGS. 1-3 illustrate the frame 20 constructed in a block configuration with the passages 25 then being formed within the block construction. Nevertheless, it is contemplated and included that frame 20 may have a variety configurations.

For example, a surgical screw is shown inserted into an alternate embodiment of the caddy in FIG. 4. The embodiment of FIG. 4 is identical to the alternate embodiment of FIGS. 1-3 with the exception that frame 20 is not constructed in block form but includes a pair of separate plates 61 and 62 spaced apart by gap 63. Thus, the rigid cover 21 is fixedly secured by fasteners 22 (FIG. 2) to rigid member 61 with flexible sheet 23 positioned between cover 21 and member 61. Fasteners 22 force cover 21 against flexible sheet 23, in turn, forced against plate 61.

Plate 61 has a hole 64 with the same diameter as counter bore 44 previously described and sized with respect to the movable flaps. Thus, the shank of the surgical screw is extended through hole 24 of cover 21 forcing the movable flaps into hole 64 allowing the movable flaps to be deflected. The shank of the screw continues downwardly through gap 63 and is received in an upwardly opening recess 65 forming a tapered stop surface 67 aligned with drain hole 28. Thus, the tip 66 of the screw shank contacts the tapered stop surface 67 of recess 65 formed in plate 62. Plates 61 and 62 are connected together by the side walls 11 and 14 and end walls 12 and 13 (FIG. 1). Thus, the embodiment of FIG. 4 is identical to the alternate embodiment with the exception that in the design of FIG. 4, a cylindrical passage does not extend between hole 64 and recess 65. Side ways movement of the screw shank is limited by the tip 66 of the shank being nested within recess 65. Plate 62 may extend in a single plane between end walls 12 and 13 providing reception of the same length of screws. Alternatively, a plurality of plates 62 may be connected together in step form in accordance with the bottom step configuration of frame 20 to accept different lengths of screws. Thus, the stop surfaces formed by an internal surface of recess 65 are located the distances from the flexible sheet 23 to position the surgical screws within the frame depending upon the length of the shank of the surgical screws in order to position the screw heads outwardly of cover 21.

In lieu of using the block configuration of FIG. 1-3 or the plate configuration of FIG. 4, the present invention includes using an entirely open design wherein one or more flexible sheets may be arranged atop one another forming a sandwich configuration to align and hold the surgical fasteners and allow the fasteners to be visible form the side of the caddy. In the sandwich configuration, the embodiment shown is FIG. 4 is modified slightly by replacing wall 62 with a pair of rigid walls between which is positioned a flexible wall all secured together and spaced apart from walls 21 and 61 and sheet 23. The flexible wall positioned between the pair of rigid walls may include flaps identical to those previously disclosed or may simply have a passage through which the fastener shank may pass.

The caddy shown in the drawings are particularly useful in combining with the plurality of surgical fasteners having shanks extended into the caddy. The fasteners may be removed one at a time from the caddy for use during surgery.

In order to indicate to the user the particular length of screw inserted into the caddy, indicia 70 is marked on the outward surface of cover 21 in a conventional fashion as is the custom with commercially available screw caddies.

The flexible sheet 21 is produced from silicone for both embodiments disclosed herein whereas cover 21, frame 20 and plates 61 and 62 are produced from a rigid material. Cover 21 is immovable and fixed to plate 61 by fasteners 22 or other conventional means.

The caddy disclosed herein will accept fastener shanks of different diameters within the same hole since the flaps will deflect as the shanks are inserted. The flaps might not return to a non-deflected condition when the shanks are pulled outwardly in the event the shanks are of sufficient large diameter relative to the spacing between the distal ends of the flaps. The flaps deflect ninety degrees into the caddy during shank insertion and remain deflected while holding the shank. The flaps reverse deflection when the shank is extracted from the caddy. On screws or implants with smooth sides or fine threads, the flaps may not need to reverse deflection.

The caddy may be utilized for holding not only surgical screws but also holding any type of cylindrical item, such as other implants including small rods or bone plates. Thus, it is to be understood that the term fastener used herein includes any type of cylindrical item including small rods or bone plates.

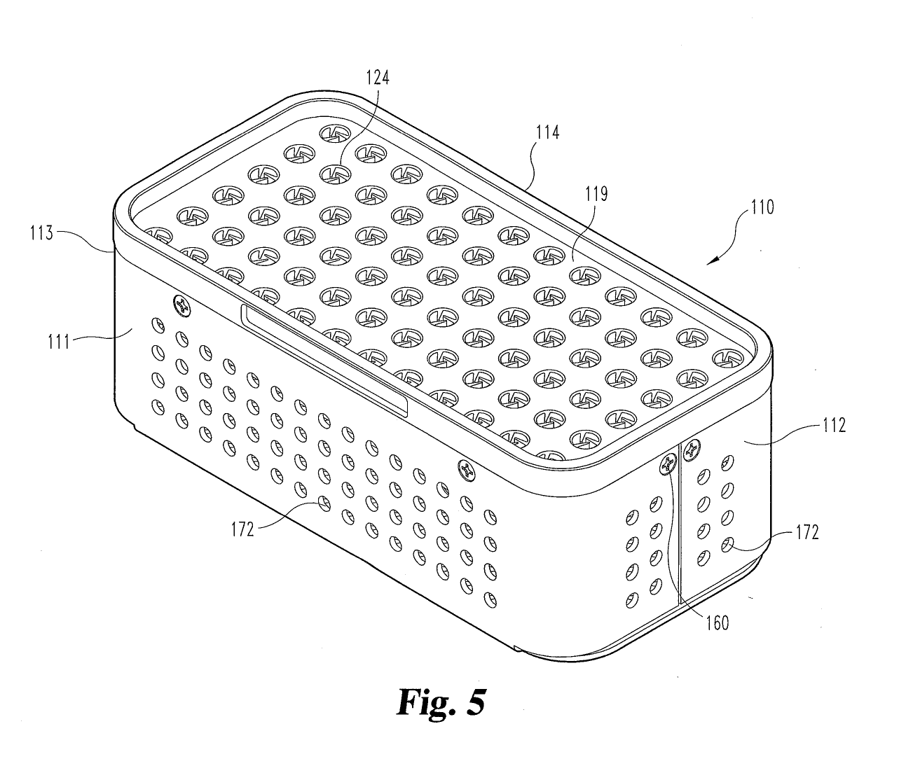

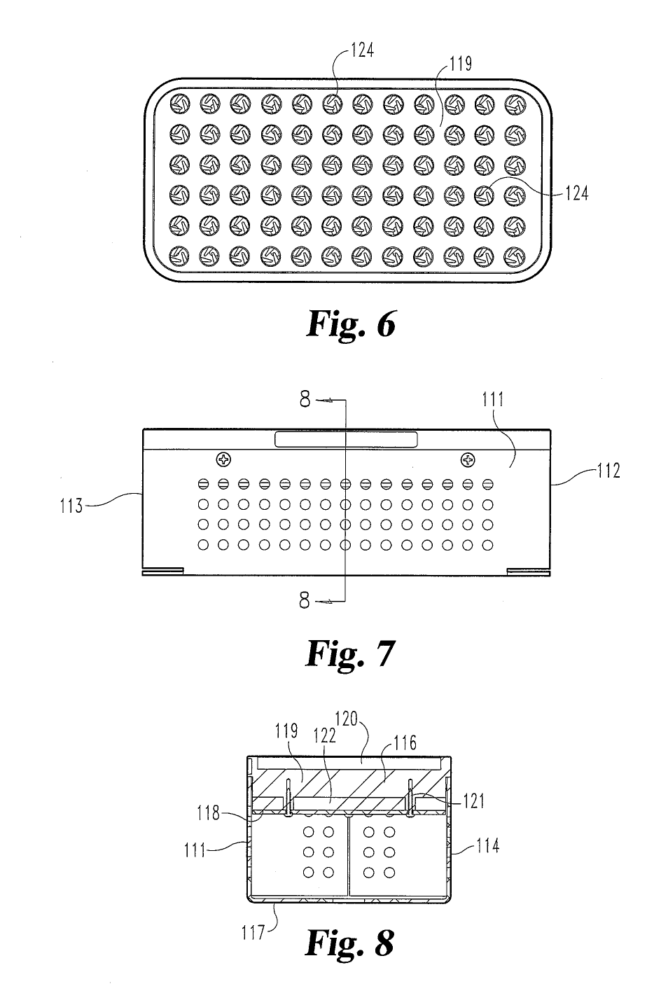

The preferred embodiment of the universal surgical fastener caddy is illustrated in FIGS. 5-13. The caddy 110 includes a box like enclosure having a pair of side walls 111 and 114 joined to end walls 112 and 113 with the end walls and side walls also joined to the bottom wall 117. An intermediate rigid wall 118 (FIG. 8) is located within the caddy and positioned beneath a flexible sheet 122, in turn, positioned beneath a top rigid cover 119. The flexible sheet may be silicone material or other suitable material. A plurality of holes 172 (FIG. 5) extend through the side walls and end walls and allow circulation of fluid during the sterilization process. Cover 119 may be secured to the caddy in a number of ways. For example, a plurality of standard fastening devices 160 (FIG. 5) may be extended through the end and side walls and into cover 119 holding the cover rigidly thereto. A plurality of conventional fastening devices 121 (FIG. 8), such as screws, are extended through intermediate member 118, through flexible sheet 122 and then into cover 119. Fastening devices 121 thereby secure intermediate member 118 to cover 119 with the flexible sheet 122 held fixedly therebetween. Cover 119 may include a recess 120 extending the width and length of the caddy between the side walls and end walls which project above the recess.

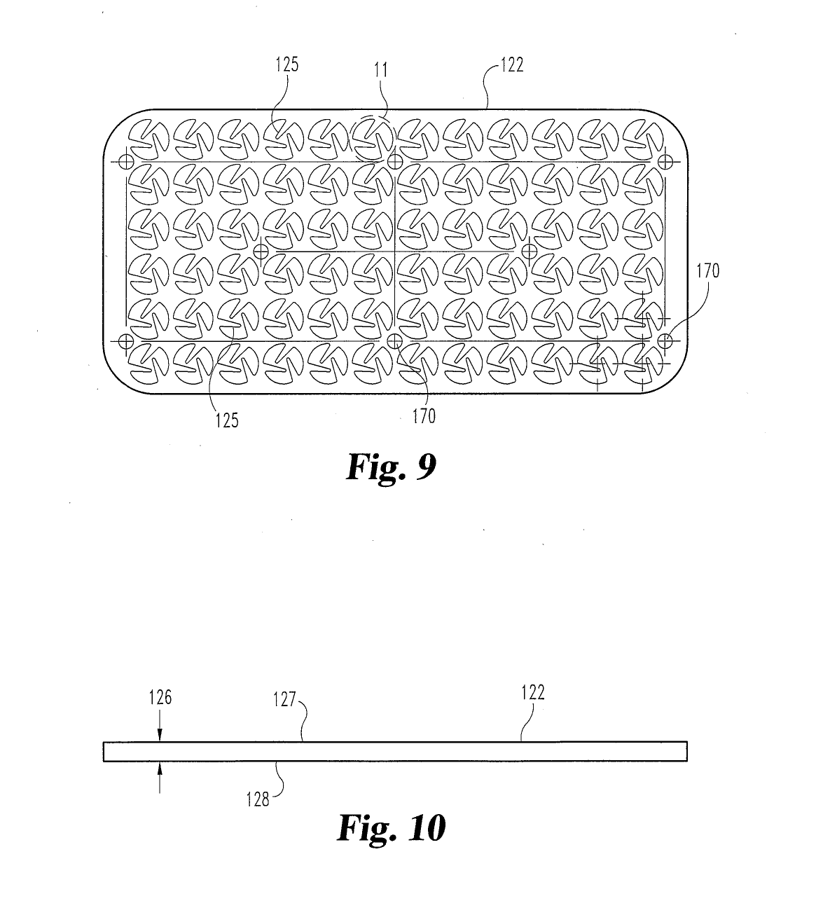

Cover 119 includes a plurality of holes 124 (FIG. 5) which extends there through with the holes being arranged across the width and length of the cover. Flexible sheet 122 (FIGS. 9 and 10) has a fixed thickness 126 extending along the length and width of the sheet with the top surface 127 and bottom surface 128 being parallel. Holes 170 (FIG. 9) extend through the sheet to allow fastening devices 121 (FIG. 8) to extend from the intermediate member 118 into cover 119.

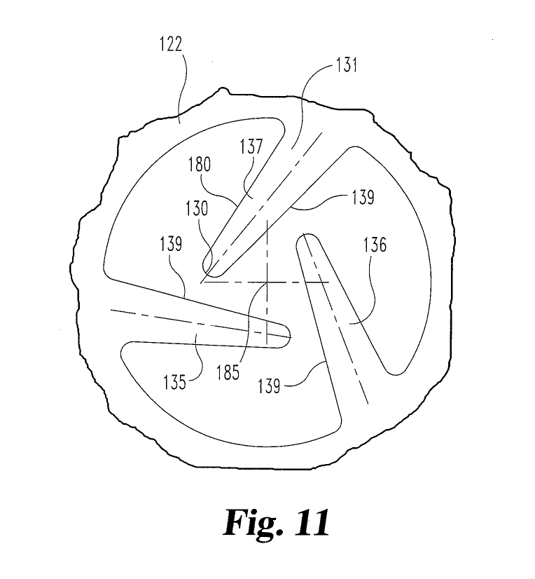

Holes 125 are formed in the flexible sheet 122 by a plurality of flaps or movable portions. Holes 125 in sheet 122 are arranged beneath holes 124 of cover 119. The holes 125 are identical in the embodiment shown in the drawings. Once such hole 125 enclosed in the dashed circle 11 of FIG. 9 will now be described it being understood that an identical description applies to the remaining holes 125. Three flexible and movable flaps 135, 136 and 137 (FIG. 11) each have a proximal end integral with sheet 122 and free and movable distal end portions. For example, flap 137 has proximal end 131 integral with sheet 122 while the distal end 130 is positioned outwardly thereof. The three flaps 135-137 define areas through which shanks of surgical implants may be extended.

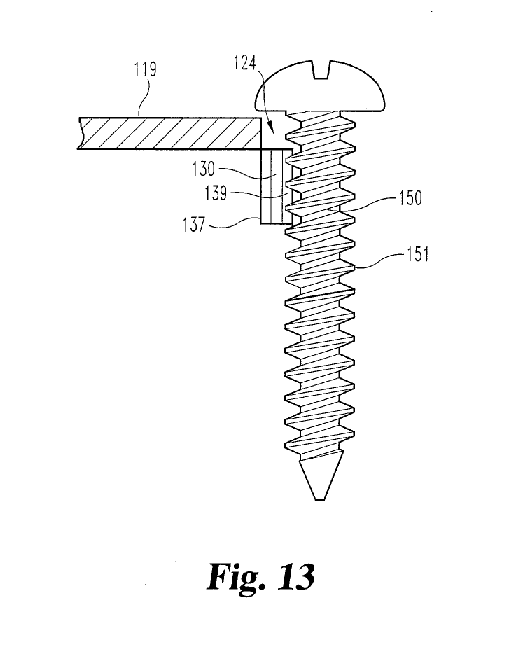

Each flap 135-137 is tapered and decreases in width from their proximal ends to their distal ends which are rounded. For example, proximal end 131 of flap 137 has a greater width than the width of the distal end 130. Each flap has a pair of opposite side surfaces that converge in a direction from the proximal end to the distal end. For example, flap 137 has a side surface 139 and a side surface 180. Side surface 139 faces flaps 135 and 136 whereas side surface 180 faces away from flaps 135 and 136 and thus, away from the passage 185 formed between the three flaps. The side surface of each flap that face toward the other flaps define the area or passage into which the surgical implant is extended. Thus, the outside screw surface 151 (FIG. 13) of implant 150 is contacted by side surface 139 of flap 137 between the flap proximal end 131 and the distal end 130. Similarly, the screw surface 151 contacts side surfaces 139 of flaps 135 and 136. The flaps are flexible and pivot clockwise as viewed in FIG. 11 about their proximal ends as the implant is inserted into passage 185 between the flaps with surfaces 139 contacting the implant shank and releasably holding the implant in place and limiting sideways movement of the shank within the caddy until the head of the implant is gripped and pulled outwardly from the caddy. Hole 124 of cover aligned over the flexible sheet hole 125 has a sufficiently large diameter to allow the shank of the implant to extend into the flexible sheet but a sufficiently small diameter to prevent the head of the implant to extend through the cover.

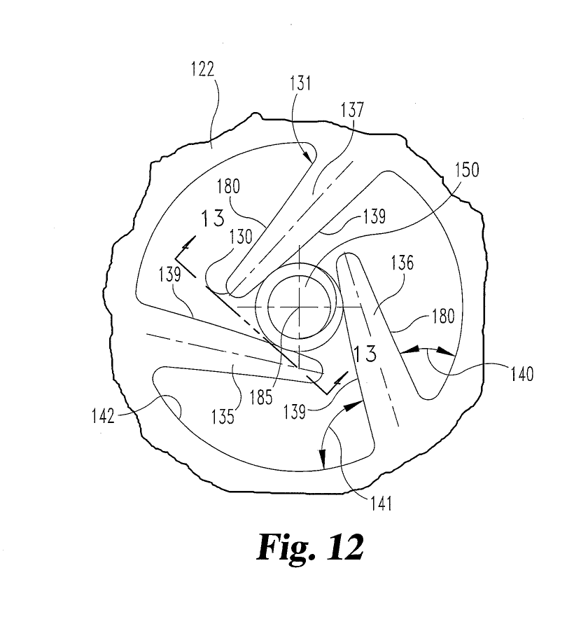

The flaps are normally in an unstressed and relaxed position depicted in FIG. 11 when the surgical implant is not extended between the flaps. Once the surgical implant is inserted between the flaps (FIG. 12), the flaps are contacted by the implant shank and flex and move further apart in a general sideways direction perpendicular to the diameter to implant shank. Acute angle 140 between surface 180 and the internal cylindrical edge 142 decreases as the flap moves from the relaxed position of FIG. 11 to the engaged position of FIG. 12. Similarly, obtuse angle 141 between surface 139 and the internal cylindrical edge 142 increases as the flap moves from the relaxed position of FIG. 11 to the engaged position of FIG. 12. The flaps have sufficient thickness equal to the thickness 126 (FIG. 10) of sheet 122 and thus do not flex in the direction of the thickness. Internal cylindrical edge is interrupted by the proximal ends of the three flaps but is continuous between flaps. Passage 185 formed by surfaces 139 defines an area arranged so the flaps are spaced apart less than the diameter of the shank of the implant thereby requiring the surfaces 139 to move further apart when the shank is inserted therebetween.

While the invention has been illustrated and described in detail in the drawings and foregoing description, the same is to be considered as illustrative and not restrictive in character, it being understood that only the preferred embodiments have been shown and described and that all changes and modifications that come within the spirit of the invention are desired to be protected.

What is claimed is:

1. A surgical implant holder for holding surgical implants having shanks with side shank surfaces comprising:
a frame;
a flexible sheet including a plurality of flaps with proximal ends integral with said sheet and free distal ends, said flaps arranged in groups defining areas in said sheet through which shanks of surgical implants may be extended with said flaps having side contact surfaces extending between said proximal ends and said distal ends with said side contact surfaces engageable with said shanks to releasably hold said shanks; and,
a cover mounted to said frame and positioned outwardly of said flexible sheet limiting outward movement of said flexible sheet when said shanks are withdrawn from said holders; and wherein:
said flaps have a relaxed position extending inwardly toward each other when said surgical implants are spaced apart from said surgical implant holder and an stressed engaged position when said surgical implants are extended between said flaps contacting said side contact surfaces releasably holding said shanks.

2. The holder of claim 1 wherein:
said side contact surfaces of said flaps for each of said flaps face inwardly defining said areas; and,
said frame has a member positioned inwardly of said flexible sheet limiting inward movement of said flexible sheet when said shanks are inserted in said frame.

3. The holder of claim 2 wherein:
said flaps each have a proximal end integral with said sheet and an outwardly located distal end and taper decreasingly from said proximal end to said distal end.

4. The holder of claim 3 wherein:
said sheet and flaps have equal thickness.

5. The holder of claim 4 wherein:
said flaps flex about each proximal end but are inflexible in the direction of said thickness.

6. The holder of claim 5 wherein:
said flaps move further apart from each other as they pivot about each proximal end moving from said relaxed position to said stressed engaged position.

7. The holder of claim 3 wherein:
said sheet has an internal cylindrical edge with said flaps arranged at an acute angle relative to said internal cylindrical edge, said acute angle decreases as said flaps move from said relaxed position to said stressed engaged position.

8. A universal surgical implant caddy comprising:
a surgical implant with a shank and shank side surface;
a housing including:
a frame;
a flexible device mounted to said frame, said flexible device has movable portions with side contact surfaces defining a passage, said movable portions have a relaxed state when said shank is apart from said housing and a stressed engaged state when said shank is extended into said passage moving said movable portions with said side contact surfaces releasably holding said shank side surface and shank; and,
a rigid cover mounted to said frame limiting movement of said movable portions when said shank is withdrawn from the caddy.

9. The caddy of claim 8 wherein:
said cover has a hole aligned with said passage sized relative to said implant to limit sideways movement of said implant; and,
said movable portions have proximal ends and free distal ends with said side contact surfaces extending between said proximal ends and said free distal ends, said movable portions pivot about said proximal ends to move said side contact surfaces further apart as they are moved from said relaxed state to said stressed engaged state.

10. The caddy of claim 9 wherein:
said surgical implants have heads located outwardly of said housing to allow the heads to be grasped when said surgical implant is inserted into said passage.

11. The caddy of claim 10 wherein:
said movable portions are tapered from said proximal ends to said distal ends.

12. The combination of:
a plurality of surgical implants having shanks;
a surgical implant caddy for holding said surgical implants during sterilization thereof and thereafter as said implants are removed, one at a time, from said caddy for use in surgery; and wherein said caddy includes:
a flexible sheet;
a member; and,
a caddy frame having a top end with said flexible sheet and member forming a plurality of passages each sized to receive said shanks, said flexible sheet held by said member and having a plurality of movable flaps integrally joined to said flexible sheet and having a first position and a second position, said shanks extend into said passages being removably held therein by said movable flaps, said movable flaps arranged in a group associated with each passage with said movable flaps in a group when in said first position spaced apart a distance less than the diameter of said shank, said movable flaps in a group moving further apart in said second position limiting movement of said shank when inserted therebetween.

12. The combination of claim 11 wherein
said surgical implants are screws with threaded shanks and heads integral therewith, said passages are sized to allow insertion of said shanks into said passages while preventing insertion of said heads therein.

13. The combination of claim 11 wherein:
said flexible sheet is silicone.

14. The combination of claim 11 wherein:
said frame has a plurality of drain holes allowing sterilization fluid to flow past said movable flaps and via said passages and then out of said frame via said drain holes.

15. The combination of claim 15 wherein:
said rigid sheet is immovable and includes holes into which said shanks extend with said holes aligned with said passages to allow grasping thereof as said shanks are pulled outwardly from said passages.

16. A caddy in combination with surgical fasteners comprising:
a plurality of surgical fasteners with cylindrical shafts;
a housing with passages to receive said cylindrical shafts; and,
a plurality of movable portions mounted to said housing, said movable portions having side surfaces with said movable portions arranged in groups, said movable portions in said groups having proximal ends fixed to said housing and freely movable distal end portions with said movable portions deflected sideways in said passages with said side surfaces which extend from said proximal ends to said distal end portions engaging and removably holding said cylindrical shafts in said passages but yieldable to allow said cylindrical shafts to be pulled outwardly from said housing.

17. The caddy in combination with surgical fasteners of claim 16 wherein:
said housing has a first rigid component with holes and a second rigid component, said plurality of movable portions are in the form of a flexible sheet located between said first rigid component and said second rigid component; and,
said surgical fasteners have enlarged heads relative to said cylindrical shafts with said holes of said first rigid component being smaller than said heads limiting movement of said heads through said holes.

18. The caddy in combination with surgical fasteners of claim 17 wherein said first rigid component is a cover and said second rigid component is an interior wall with said flexible sheet located between said cover and said interior wall.
